(19) United States
(12) Patent Application Publication
(10) Pub. No.: US 2020/0261722 A1
(43) Pub. Date: Aug. 20, 2020

(54) PERIPHERAL NERVE STIMULATION DEVICE FOR AFFECTING PARASYMPATHETIC AND SYMPATHETIC ACTIVITY TO ACHIEVE THERAPEUTIC EFFECTS

(71) Applicant: Vorso Corp., Redwood City, CA (US)

(72) Inventors: Konstantinos Alataris, Menlo Park, CA (US); Vivek Sharma, San Ramon, CA (US); Gary Heit, La Honda, CA (US)

(21) Appl. No.: 16/723,833

(22) Filed: Dec. 20, 2019

Related U.S. Application Data
(63) Continuation of application No. PCT/US2018/039467, filed on Jun. 26, 2018.
(60) Provisional application No. 62/576,440, filed on Oct. 24, 2017.
(60) Provisional application No. 62/525,151, filed on Jun. 26, 2017.

(51) Int. Cl. A61N 1/36; A61N 1/04; A61K 45/06

(52) U.S. Cl. CPC A61N 1/36034; A61N 1/0456; A61N 1/36036; A61N 5/0622; A61N 1/36031; A61N 1/36021; A61K 45/06

(57) ABSTRACT

The present disclosure relates to devices and methods for stimulating peripheral nerves in a patient via electrical, optical, mechanical, or other stimulation, in order to change the balance between parasympathetic and sympathetic activity by selectively increasing or decreasing each of parasympathetic and sympathetic activity. In a particular application, the present disclosure relates to a device for transdermal stimulation of the vagus nerve (including the auricular branch) to selectively affect the sympathetic and parasympathetic nervous system to achieve the desired therapeutic effect in a human subject.

### CROSS-REFERENCE TO RELATED APPLICATION(S)

This application is a continuation of International Application No. PCT/US2018/039467 filed on Jun. 26, 2018, which claims priority to U.S. Provisional Application Ser. No. 62/576,440 filed on Oct. 24, 2017 and U.S. Provisional Application Ser. No. 62/525,151 filed on Jun. 26, 2017, which are incorporated herein by reference in their entirety to the full extent permitted by law.

### TECHNICAL FIELD

The present invention relates to the field of neurostimulation for the treatment of one or more conditions and to the field of stimulation of a peripheral nerve to achieve therapeutic effects. The invention includes methods and devices for providing transcutaneous electrical stimulation of a vagus nerve of a patient through one or more structures of the ear of a patient. More particularly, the present invention relates to devices and methods for stimulating peripheral nerves in a patient via electrical, optical, mechanical, or other stimulation, in order to change the balance between parasympathetic and sympathetic activity by selectively increasing or decreasing each of parasympathetic and sympathetic activity. Possible peripheral nerves that may be used for stimulation (one at a time or in combination) according to the techniques described herein include, but are not limited to, the vagus, auricular branch of vagus, optic, tibial, saphenous, radial or ulnar nerve. Other target nerves may be used based on further disease-state selection for application of the invention. Also, modes of stimulation include, but are not limited to, electrical stimulation, light stimulation, mechanical stimulation, and magnetic field stimulation. This stimulation may be achieved transcutaneously or via implantable stimulation delivery tools. Selection of targets can be determined by the pathophysiology dictating a modulation of either or both arms of the autonomic nervous system—parasympathetic or sympathetic branches—in a variety of situations.

### BACKGROUND

Electrical stimulation for the treatment of medical conditions has been used for many decades. Cardiac pacemakers are one of the earliest and most widespread examples of electrical stimulation to treat medical conditions, with wearable pacemakers dating from the late 1950s and early 1960s.

More recently, electrical stimulation of the brain with implanted electrodes (deep brain stimulation) has been approved for use in the treatment of various conditions, including pain and movement disorders such as essential tremor and Parkinson's disease. Electrical stimulation of the spinal cord to treat chronic pain has also become widespread since the early 2000s.

Most relevant to the present invention is electrical stimulation of the vagus nerve, which has been widely used since the late 1990s for the treatment of epilepsy and has been approved for the treatment of clinical depression since 2005. Such treatments, however, generally require a surgical procedure to attach electrodes directly to the vagus nerve in the patient's neck, which is coupled via a lead wire to a pulse generator implanted in the chest of the patient. Current VNS therapies usually involve providing an electrical signal characterized by a number of parameters including a pulse frequency, a pulse width, a current or voltage amplitude, an ON time (during which pulses at the defined frequency are applied to the target nerve) and an OFF time (during which no electrical pulses are applied to the target nerve). In some instances, longer therapy delivery periods such as 3, 4, 6, 12, or 24 hours or more are used, with the therapy being applied according to the ON time and OFF time or with no therapy being applied for a defined non-therapy period.

Transcutaneous or transdermal electrical stimulation of peripheral nerves could play a significant role in the physiologic functions of multiple organs and even have more broader implications throughout the body. The latter is due to potential changes in the processing of information by the central nervous system (CNS). Peripheral nerves not only sense and transmit information to the CNS from the periphery but also deliver signals from the CNS to the periphery to control function of organs. Somatic peripheral nerves have both afferent and efferent fibers. Afferent fibers transmit information to the CNS while the efferent fibers relay control commands from the CNS to the periphery. Peripheral nerves play a key role in both sympathetic and parasympathetic activity of the autonomic nervous system. Additionally, both branches of the autonomic nerves system (sympathetic and parasympathetic) can carry information to and from the CNS and thereby contribute to the modulation of neural networks that directly regulate specific organ functions.

Transcutaneous or transdermal stimulation of the auricular branch of the vagus nerve through the skin with electrical impulses in areas of the outer ear plays a significant role in modulating physiologic functions throughout the body. The vagus nerve (tenth cranial nerve) is the nerve that innervates many organs and through autonomic afferent and efferent fibers not only senses but also controls multiple body functions. This control is achieved via a balance with the sympathetic and parasympathetic branches of the autonomic nervous system. The vagus nerve and associated Nucleus and Tractus Solitarius (NTS) have principal roles in the control of parasympathetic activity. The sympathetic nervous system has its principal outflow nucleus in the intermediolateral horns of the spinal cord and then the preganglionic sympathetic chain. The proximal controlling CNS structure of the sympathetic nerve is thought to be the rostral lateral ventral medullar nucleus (RVLN). The vagus nerve has indirect and possibly direct axonal connections to the RVLN; both contra-lateral and ipsilateral. Auricular stimulation can neuromodulate the neural processes related to neurotransmitters such as norepinephrine, gamma-aminobutyric acid (GABA) and acetylcholine and change the parasympathetic or sympathetic activity depending on the stimulation site. Additional sites of modulation, including those that are more rostral portions, include the locus cereuleous, nuclues accumbens, elements of the hypothalamus insula, dorsal lateral, and medial orbital frontal cortices, and the cingulate cortex.

Vagus nerve stimulation was initially proposed as a therapy for epilepsy and other motor disorders by Zabara. For example, in U.S. Pat. No. 4,702,254 (and related U.S. Pat. Nos. 4,867,164, and 5,025,807), low frequency stimulation of the vagus nerve is proposed to treat epilepsy, seizures, cerebral palsy, and Parkinson's disease. In particular, stimulation of the vagus nerve is proposed using a pulsed electrical signal having a pulse frequency of from 30 to 300 Hz, a pulse width of 300 to 1000 microseconds, and with a constant current of from 1 to 20 mA. Treatment of numerous other conditions with VNS has been proposed by Terry, Jr., and others for neuropsychiatric disorders such as depression (U.S. Pat. No. 5,299,569), migraine headaches (U.S. Pat. No. 5,215,086), endocrine disorders (U.S. Pat. No. 5,231,988), eating disorders (U.S. Pat. No. 5,263,480), dementia (U.S. Pat. No. 5,269,303), pain (U.S. Pat. No. 5,330,515), sleep disorders (U.S. Pat. No. 5,335,657), motility disorders (U.S. Pat. No. 5,540,730), hypertension (U.S. Pat. No. 5,707,400), obesity (U.S. Pat. No. 6,587,719), heart failure (U.S. Pat. No. 6,622,041), and many other conditions. Each of the patents referred to in this paragraph is hereby incorporated by reference in its entirety.

The foregoing patents all involve electrical stimulation of the vagus nerve at relatively low frequencies, usually below 100 Hz (20 Hz to 30 Hz are common therapies for VNS for the treatment of epilepsy), but occasionally extending as high as 300 Hz. Low frequency VNS is believed to result in the induction of afferent or efferent action potentials on the nerve to a target organ (i.e., the brain for afferent stimulation, or the stomach, intestines, lungs, pancreas, liver, or other organs for efferent stimulation). At higher frequencies, (usually referred to as above 500 Hz), it is generally believed that the stimulation signal effectively precludes action potentials from passing through the stimulation site, i.e., that high frequency stimulation creates a conduction block on the vagus nerve at the stimulation site that prevents nerve impulses (action potentials) from crossing the stimulation site.

The conduction blocking effect of high-frequency stimulation, sometimes referred to as a “reversible vagotomy,” has been incorporated into proposed therapies for eating disorders and other gastrointestinal conditions. For example, in U.S. Pat. No. 7,167,750, incorporated by reference in its entirety, electrical stimulation of the vagus nerve at conduction-blocking frequencies of 500 to 5000 Hz was proposed as a treatment for obesity. In the same patent, lower frequency VNS at 12 Hz, referred to as “stimulation” or “pacing” frequency, was proposed to enhance vagal tone.

Research in the last twenty years suggests that VNS has anti-inflammatory effects. In particular, VNS has been proposed as a treatment for diseases mediated by pro-inflammatory cytokines such as TNF-α, IL-1α, IL-Iβ, IL-6, IL-8, IL-18, interferon-y, and many others. Inflammation may be induced by these and other pro-inflammatory cytokines, which are produced by various cell types. Inflammatory cytokines contribute to numerous conditions, including many cancers and tumors, autoimmune disorders, diseases of the musculoskeletal system, diseases of the central or peripheral nervous system, cardiovascular diseases, dermatological diseases, certain infectious diseases, respiratory diseases, gastrointestinal diseases, and many diseases characterized by local or systemic inflammation.

The use of VNS to reduce pro-inflammatory cytokine production has been proposed in U.S. Pat. No. 8,914,114 Tracey et al., and other related patents (e.g. U.S. Pat. Nos. 6,610,713, 8,391,970, 8,729,129, 9,211,409, and 9,662,490), each of which is hereby incorporated by reference in its entirety. These patents describe the use of efferent VNS to reduce the release of inflammatory cytokines from mammalian cells to inhibit conditions or diseases mediated by inflammatory cytokine cascades. The precise stimulation parameters affecting the release of pro-inflammatory cytokines is the subject of ongoing research.

In addition to the pro-inflammatory cytokines previously noted, other cytokines are known to have anti-inflammatory effects. These include IL-4, IL-6, IL-10, IL-11, and IL-13. In addition, specific cytokine receptors for IL-1, TNF-α, and IL-18 also function as pro-inflammatory cytokine inhibitors. While reduction of pro-inflammatory cytokines may have beneficial effects on diseases mediated by such cytokines, it is undesirable to reduce anti-inflammatory cytokines. There is a need for therapies that can both reduce pro-inflammatory cytokines and increase (or at least not reduce) anti-inflammatory cytokines.

Non-surgical VNS, including stimulation of the skin at the neck or stimulation of the auricular branches of the vagus nerve through the ear, have been proposed, but the interfaces for delivering the stimulation have been bulky and difficult to maintain in contact with the patient's skin. In addition, external stimulation (i.e., applying the electrical signal from the outside of the patient's body) across the skin presents a more difficult challenge than surgically implanted electrodes in direct contact with the vagus nerve.

In implanted VNS systems, the direct electrode-nerve coupling allows the electrical signal to be delivered to the nerve with a high degree of consistency and fidelity, since the electrodes maintain the same position over time and there is no attenuating tissue between the electrode and the nerve fibers. In contrast, transcutaneous VNS systems must overcome the electrical resistance and current attenuation of the patient's skin (which may vary in thickness, elasticity, etc. from patient to patient) as well as differences in anatomical position of the vagus nerve under the skin. Although the general locations of vagus nerve branches within the ear are known, the precise location of the vagus nerve cannot be known for a particular patient in transcutaneous VNS. For this reason, many proposed external VNS systems either misalign the electrodes such that little or no electrical current is actually delivered to the nerve, or the electrode holder may shift position over time or with patient movement, such that delivery of the current to the nerve target is unreliable or episodic. Finally, the resistivity of the skin varies over time, even for a particular patient, based on sweat, oils, and/or wax secreted by the skin.

Transcutaneous vagus nerve stimulation (“tVNS”) has typically involved the use of a stimulation unit and direct transcutaneous vagal nerve stimulation. Treatment sessions have varied from about an hour per day to 3 to 4 sessions of an hour or longer each per day. The tVNS has been used to treat a variety of disorders. For example, tVNS has been used in attempts to treat epilepsy, anxiety, depression, other neuropsychiatric disorders, and other diseases. A number of devices have been proposed to deliver tVNS as, for example, described in the following: U.S. Pat. Nos. 7,797,042; 8,688,239; 8,666,502; 8,885,861; 9,339,641; U.S. Patent Application Publication No. 20100057154; U.S. Patent Application Publication No. 20130079862; U.S. Patent Application Publication No. 20150165195; and U.S. Patent Application Publication No. 20160022987. Other devices are available from Nervana, Cerbomed and ElectroCore.

However, prior devices and methods have a number of disadvantages, including, for example, lacking the ability to effectively treat disease or up-regulate or down-regulate the afferent and/or efferent traffic or impact both the sympathetic and parasympathetic activity in a coordinated way. Previous devices are also prone to untoward side effects such as paresthesia and might include buzzing, tingling, hoarseness, shortness of breath, change of voice during treatment, bradycardia, or other detectable and potentially uncomfortable sensations while the device is on. These untoward side effects and paresthesias may limit patient compliance. These paresthesias also may contaminate the claim of parasympathetic modulation.

Accordingly, there is a need for improved systems for delivery of transcutaneous vagus nerve stimulation that are compact, light, comfortable for the patient, consistently positionable in the same location, and able to consistently deliver electrical current over a relatively wide area to accommodate anatomical differences. In addition, there is a need for a device that can be used to stimulate the transcutaneous peripheral nerve to achieve a desired therapeutic effect in a human subject.

### SUMMARY

The present invention relates to an electrical stimulation apparatus for providing a neurostimulation signal to a target portion of an ear of a patient, comprising: a first, generally cylindrical interface member having a C-shaped cross-section, wherein the external periphery of the C-shape is adapted to engage a target portion of a left or a right ear of the patient; at least one first electrode coupled to the external periphery of the interface member, the at least one first electrode adapted to contact the skin of the target portion of the left or right ear and to deliver a first electrical signal transcutaneously to a neural structure proximate the target portion; and a first electrical stimulation module, coupled to the at least one first electrode, adapted to generate and apply a first electrical signal to the at least one first electrode, the first electrical stimulation signal comprising a pulsed electrical signal having a frequency of from 1 Hz to 50 kHz, a pulse width of from 1-500 microseconds, and a current of from 1 mA to 20 mA.

In one embodiment, the invention relates to an electrical stimulation apparatus for providing a neurostimulation signal to a target portion of an ear of a patient, comprising: a first interface member having a C-shaped cross-section, wherein the external periphery of the C-shape is adapted to engage a target portion of a left or a right ear of the patient; at least one first electrode coupled to the external periphery of the interface member, the at least one first electrode adapted to contact the skin of the target portion of the left or right ear and to deliver a first electrical signal transcutaneously to a neural structure proximate the target portion; and a first electrical stimulation module, coupled to the at least one first electrode, adapted to generate and apply a first electrical signal to the at least one first electrode, the first electrical stimulation signal comprising a pulsed electrical signal having a frequency of from 1 Hz to 50 kHz, a pulse width of from 1-500 microseconds, and a current of from 1 mA to 20 mA.

In another embodiment, the invention relates to an electrical stimulation apparatus for providing a neurostimulation signal to a target portion of an ear of a patient, comprising: a first, generally cylindrical interface member, wherein the external periphery of the interface member is adapted to engage a target portion of a left or a right ear of the patient; at least one first electrode coupled to the external periphery of the interface member, the at least one first electrode adapted to contact the skin of the target portion of the left or right ear and to deliver a first electrical signal transcutaneously to a neural structure proximate the target portion; and a first electrical stimulation module, coupled to the at least one first electrode, adapted to generate and apply a first electrical signal to the at least one first electrode, the first electrical stimulation signal comprising a pulsed electrical signal having a frequency of from 1 Hz to 50 kHz, a pulse width of from 1-500 microseconds, and a current of from 1 mA to 20 mA.

In yet another embodiment, the invention relates to an electrical stimulation system for providing a neurostimulation signal to a target portion of an ear of a patient, comprising: a first interface member having an external periphery adapted to engage a target portion of a left or a right ear of the patient; at least one first electrode comprising an external periphery of the interface member, the at least one first electrode adapted to contact the skin of the target portion of the left or right ear and to deliver a first electrical signal transcutaneously to a neural structure proximate the target portion; at least one electrical stimulation module, coupled to the at least one first electrode, adapted to generate and apply a first electrical signal to the at least one first electrode, the first electrical stimulation signal comprising a high frequency pulsed electrical signal having a frequency of from 1 kHz to 50 kHz, a pulse width of from 1-500 microseconds, and a current of from 1 mA to 20 mA.

In another embodiment, the invention teaches a method of providing a neurostimulation therapy to a neural structure in the ear of a patient, comprising: generating a high frequency pulsed electrical signal comprising a pulse frequency of from 1 kHz to 50 kHz, a pulse width of from 1-500 microseconds, and a current of from 1 mA to 20 mA; and applying the high frequency pulsed electrical signal to the skin of a target portion of the ear of the patient proximate to a neural structure in the ear of the patient.

In one embodiment, the invention teaches a method of providing a neurostimulation therapy to a neural structure in the ear of a patient, comprising: generating a pulsed electrical signal comprising a pulse frequency of from 5 Hz to 50 kHz, a pulse width of from 1-500 microseconds, and a current of from 1 mA to 20 mA; and applying the pulsed electrical signal to the skin of a target portion of the ear of the patient proximate to a neural structure in the ear of the patient so as to reduce at least one pro-inflammatory biomarker and increase at least one anti-inflammatory biomarker.

In yet another embodiment, the invention provides a method of providing a neurostimulation therapy to a plurality of neural structures in an ear of a patient, comprising: generating a first high frequency pulsed electrical signal comprising a pulse frequency of from 3 kHz to 50 kHz, a pulse width of from 1-500 microseconds, and a current of from 1 mA to 20 mA; applying the first high frequency pulsed electrical signal to the skin of a first target portion of an ear of the patient proximate to a first neural structure in the ear of the patient, the first high frequency pulsed electrical signal having at least one effect selected from an increase in the patient's parasympathetic tone, a decrease in the patient's sympathetic tone, an increase in at least one anti-inflammatory biomarker, and a decrease in at least one proinflammatory biomarker; generating a second high frequency pulsed electrical signal comprising a pulse frequency of from 3 kHz to 50 kHz, a pulse width of from 1-500 microseconds, and a current of from 1 mA to 20 mA; and applying the second high frequency pulsed electrical signal to the skin of a second target portion of an ear of the patient proximate to a second neural structure in the ear of the patient, the second high frequency pulsed electrical signal having at least one effect selected from an increase in the patient's parasympathetic tone, a decrease in the patient's sympathetic tone, an increase in at least one anti-inflammatory biomarker, and a decrease in at least one proinflammatory biomarker, wherein the effect of the second high frequency pulsed electrical signal is different from the effect of the first high frequency pulsed electrical signal.

In an embodiment, the current invention teaches a method of providing a neurostimulation therapy to a plurality of vagus nerve structures in the body of a patient, comprising: generating a first high frequency pulsed electrical signal comprising a pulse frequency of from 3 kHz to 50 kHz, a pulse width of from 1-500 microseconds, and a current of from 1 mA to 20 mA; applying the first high frequency pulsed electrical signal to a first vagus nerve structure of the patient, the first high frequency pulsed electrical signal having at least one effect selected from an increase in the patient's parasympathetic tone, a decrease in the patient's sympathetic tone, an increase in at least one anti-inflammatory biomarker, and a decrease in at least one pro-inflammatory biomarker; generating a second high frequency pulsed electrical comprising a pulse frequency of from 3 kHz to 50 kHz, a pulse width of from 1-500 microseconds, and a current of from 1 mA to 20 mA; and applying the second high frequency pulsed electrical signal to a second vagus nerve structure of the patient, the second high frequency pulsed electrical signal having at least effect selected from an increase in the patient's parasympathetic tone, a decrease in the patient's sympathetic tone, an increase in at least one anti-inflammatory biomarker, and a decrease in at least one pro-inflammatory biomarker, wherein the effect of the second high frequency pulsed electrical signal is different from the effect of the first high frequency pulsed electrical signal.

Furthermore, the present disclosure relates to a novel device for nerve stimulation, which permits an efficient stimulation of the autonomic nervous system, specifically during a patient's daily routine and can do so in an unobtrusive way. In one embodiment, the device does not cause paresthesia (buzzing, tingling, etc.) or uncomfortable sensations while the device is on, or any stimulation-induced feeling, and is imperceptible to the user. The device is safe, non-invasive, easy to use, comfortable and can be removed quickly from the body as desired. Applications of the present disclosure include, but are not limited to vagal/auricular stimulation, stimulation of tibial nerve, radial or ulnar nerve and/or a combination of those stimulation points. Other nerves may be targeted for treating a variety of diseases or conditions. Selection of targets can be determined by the pathophysiology dictating a modulation of either, or both arms of the autonomic nervous system, i.e., the parasympathetic or sympathetic branches in a variety of transcutaneous situations.

In one embodiment, the devices and methods for stimulation of a nerve (or multiple nerves in combination) to achieve the desired effect on parasympathetic and sympathetic activity are adapted such that the patient does not feel an indication that stimulation is occurring by choosing a range of operative frequencies that would not be detected by the patient. In some embodiments, frequencies in excess of 5,000 Hz are used, or frequencies in excess of 20,000 Hz are used for this purpose. Stimulation parameters are adjusted either in an open loop fashion or in a closed loop fashion based on a sensed signal.

In one embodiment, the present invention relates to a device for transdermal stimulation of a or multiple peripheral nerves in a human subject, comprising: (i) a control unit capable of generating an electric current, (ii) a housing connected to the control unit and designed to be fitted on or in each of the human ears comprising at least one stimulation electrode to provide a stimulation current to the ear, and (iii) at least one reference electrode, wherein the device is capable of modulating both afferent and efferent fibers via electrical current and selectively modulating (upregulating or downregulating) the sympathetic system and/or the parasympathetic side. Also, stimulation patterns (including location, duration and waveforms) can be controlled based on an indication of efficacy or reduction in medication-related side effects. Further, the device of the present disclosure may, in a controlled fashion, induce up- or down-regulation of sympathetic or parasympathetic activity separately in order to rebalance or change the balance between sympathetic and parasympathetic activity.

In another embodiment, the control unit is integral to the housing. Additionally or alternatively, the control unit may be separated from the housing and connected by a wired or wireless connection. In one embodiment, the device optionally includes functionality for biometric authentication and/or patient self-assessment.

In other embodiments, the device is useful in treating a disease or condition in combination with a therapeutic agent such as a pharmaceutical. The combination or singular use of transcutaneous peripheral nerve stimulation to modulate the autonomic nervous system by affecting the sympathetic (SYMP) and/or parasympathetic (PSYMP) activity (meaning can be either combination of up-regulation or down-regulation of the two sides of the autonomic system) and the therapeutic agent results in an additive effect in treating the disease. In another embodiment, the effect is synergistic and lowers the amount of pharmaceutical needed for effective treatment of the disease.

Further, the present disclosure contemplates a method of treating rheumatoid arthritis in a human subject through the use of the device described above, comprising the steps of: administering, through the use of the device described above, transdermal stimulation of the vagus nerve or a branch of the vagus nerve (i.e., auricular) to modulate the autonomic response by affecting the sympathetic and/or parasympathetic activation; and administering an effective amount of a pharmaceutical selected from the group consisting of, but not limited to, methotrexate, abatacept, adalimumab, adalimumab-atto, anakinra, certolizumab, etanercept, etanercept-szzs, golimumab, infliximab, infliximab-dyyb, rituximab, tocilizumab, tofacitinib, and a nonsteroidal anti-inflammatory drug (NSAID).

In another embodiment, the device is used to treat asthma via a method that comprises measuring the forced expiratory volume (FEV) or nitric oxide (NO) in the subject or the response to a challenge test (like methacholine challenge test) and then adjusting the level of stimulation through the device described above based on these measurements. In other embodiments, the present disclosure includes a method for treating irritable bowel disease (IBD), sepsis or multiple sclerosis.

Other therapeutic uses of the present invention comprise treating hypertension (particularly, uncontrolled hypertension), inflammation after stroke, myocardial infarction recovery, anesthesia-induced inflammatory response, influenza, atrial fibrillation and/or relapse from cardio-conversion, sepsis, ventricular and supraventricular arrhythmias, autoimmune-mediated glomerulonephritis, Berger's IgA nephropathy, demyelination syndromes (e.g., multiple sclerosis, Devic's syndrome etc.), severe allergic reactions (e.g., skin, lungs), and autoimmune diseases (e.g., pancreatitis, gastritis, thyroiditis, hemolytic anemia, encephalitis, myasthenia gravis).

In yet another embodiment, the present invention can be used to improve the quality of sleep and to treat non-rapid eye movement (NREM) sleep disorder. Such sleep disturbances are common among elderly and Alzheimer's disease or Parkinson's disease patients. In other embodiments, the present disclosure includes a method for detecting and quantifying these sleep disturbances. In other embodiments, the present disclosure includes a method for treating migraine acutely or reducing the incidence of migraine headaches and cluster headaches. The systems and methods disclosed herein can further be used in any of the following therapeutic areas:
1. Exercise induced restrictive airway disease.
2. Topical dermatitis (e.g., poison oak, poison ivy, etc.)
3. Allergic rhinitis managed with OTCs
4. Recurrent/relapsed post-cardioversion AFIB
5. Arthropodia dermatitis (mosquito bites, tick bites, etc.)
6. Bladder/bowel control (supplement/replace anticholonergic meds)
7. Recurrent orthostatic hypertension
8. Peripheral vascular disease-Reynoud's, diabetic vasculopathy
9. Microvascular angiopathies-radiation induced
10. Early stages of inflammatory mediated nociceptive pain
11. Early stages of inflammatory mediated neuropathic pain
12. Mild food allergies
13. Solar allergies
14. Migraine headaches

In some embodiments, the device provides a current with frequency between about 0.01 Hz and 50 Hz, or between about 1 Hz and 40 Hz, or between about 5 Hz and 30 Hz, or between about 10 Hz and 20 Hz or between 5 Hz and 50 kHz or between about 1 kHz and 50 kHz, or between about 1 kHz and 10 kHz or between about 5 kHz and 10 kHz, or between about 5 kHz and 20 kHz or between about 10 kHz to 50 kHz or a combination of multiple frequencies from those ranges.

In yet other embodiments, the device provides a stimulation current between about 0.01 mA and 50 mA, or between about 1 mA and 40 mA, or between about 1 mA and 5 mA, or between 5 mA and 30 mA, or between about 10 mA and 20 mA or between 5 mA and 50 mA or between about 1 mA and 50 mA, or between about 1 mA and 10 mA or between about 5 mA and 10 mA, or between about 0.1 mA and 20 mA or a combination of multiple frequencies from those ranges.

In other embodiments, the device may use a fixed frequency or a combination of frequencies in the kHz range coupled with amplitude modulation to achieve effective autonomic regulation. Other embodiments also include kHz-weighted Gaussian frequency applications, white noise or pink noise kHz weighted stimulation frequencies or randomized kHz frequency stimulation with proscribed center weight distribution.

Additional embodiments of the present devices, methods and the like will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment or aspect. Additional aspects and embodiments are set forth in the following description and claims, particularly when considered in conjunction with the accompanying examples and drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing features of embodiments will be more readily understood by reference to the following detailed description, taken with reference to the accompanying drawings, in which:

FIGS. 1A-1E illustrate representative embodiments of devices in accordance with the present disclosure, including a control unit, housing and electrodes (FIG. 1A), a housing having two electrode positions and located over one of the patient's ears (FIG. 1B) and separately from the patient (FIG. 1C), and a housing having four electrode positions and located over one of the patient's ears (FIG. 1D) and separately from the patient (FIG. 1E), wherein the same configurations can be used in the patient's other ear.

FIG. 2 is an illustration of one embodiment of an integrated unit of the present disclosure. This configuration shows wires going to the electrodes in the cymbae.

FIG. 3 is an illustration of one embodiment of the electrodes of the present disclosure.

FIGS. 4A-4C are different views of the electrodes of the present disclosure, with FIGS. 4A and 4C illustrating isometric views, and FIG. 4B illustrating a side view.

FIG. 5 is an illustration of one embodiment of the electrode which is flexible to comply with the cymbae conchae anatomy.

FIGS. 6A-6E illustrate several views of the electrode which is flexible to comply with the cymbae conchae anatomy.

FIG. 7 is an illustration of one embodiment of the electrode of the present disclosure having a conductive sheet.

FIGS. 8A-8F illustrate an embodiment of an integrated unit of the present disclosure.

FIG. 9 is an illustration of one embodiment of the control unit of the present disclosure. The control unit (1) has a contour shape (9) that matches with anatomy of the ear. It may include an on/off switch (9), an electrode which stimulates the backside of the ear, or a photoplethysmography (PPG) system. The two circles (10) represent the transmitter and receiver of the PPG system. The electrode can be located in section (11) of the control unit or a portion of this section.

FIG. 10 is an illustration of one embodiment of the optical nerve stimulator.

FIG. 11 is a profile view of one embodiment of an interface core for an electrical stimulation interface suitable for engaging a target portion of an ear of a patient.

FIG. 12 is a block diagram of one embodiment of an electrical stimulation interface with electrodes, suitable for engaging a target portion of an ear of a patient.

FIG. 13 is a rear view of the electrical stimulation interface of FIG. 12.

FIG. 14 is a side view of an ear of a patient, with the electrical stimulation interface of FIG. 12 positioned in a cymba concha of the patient's ear.

FIG. 15 is a perspective view of one embodiment of an electrical stimulation module for use in an electrical stimulation system for providing a neurostimulation signal to a target portion of an ear of a patient.

FIG. 16 illustrates one embodiment of an electrical stimulation system, coupled to an ear of a patient, for providing a neurostimulation signal to a target portion of an ear of a patient.

FIG. 17 illustrates a rear view of the system of FIG. 16.

FIG. 18 illustrates a front view of the system of FIG. 16.

### DETAILED DESCRIPTION

The current disclosure relates to systems and methods for providing an electrical neurostimulation therapy. A generally cylindrical interface member having a C-shaped cross-section engages a target portion of a patient's ear. At least one electrode on an external periphery of the interface member contacts the target portion, and an electrical stimulation module coupled to the electrode applies a pulsed electrical signal transcutaneously to a neural structure adjacent the target portion of the ear.

The various aspects and embodiments will now be fully described herein. These aspects and embodiments may, however, be embodied in many different forms and should not be construed as limiting; rather, these embodiments are provided so the disclosure will be thorough and complete, and will fully convey the scope of the present subject matter to those skilled in the art. All publications, patents and patent applications cited herein, whether supra or infra, are hereby incorporated by reference in their entirety.

Exemplary embodiments of the present disclosure are illustrated in reference to the Figures, which are illustrative rather than restrictive. No limitation on the scope of the technology or on the claims that follow is to be implied or inferred from the examples shown in the drawings and discussed here.

### Definitions

Unless defined otherwise, all terms and phrases used herein include the meanings that the terms and phrases have attained in the art, unless the contrary is clearly indicated or clearly apparent from the context in which the term or phrase is used. Although any methods and materials similar or equivalent to those described herein can be used in the practice or testing of the present invention, particular methods and materials are now described.

Unless otherwise stated, the use of individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the disclosed subject matter is most closely related or the art relevant to the range or element at issue. The amount of broadening from the strict numerical boundary depends upon many factors. For example, some of the factors that may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value or range. Thus, as a general matter, “about” or “approximately” broaden the numerical value. Also, the disclosure of ranges is intended as a continuous range including every value between the minimum and maximum values plus the broadening of the range afforded by the use of the term “about” or “approximately.” Consequently, recitation of ranges of values herein are merely intended to serve as a shorthand method of referring individually to each separate value falling within the range, and each separate value is incorporated into the specification as if it were individually recited herein.

The term “peripheral nerve” as used herein refers to a nerve that transmits signals between the central nervous system and other body parts.

“Biometric Authentication” as used herein means biometric technologies that digitally capture fingerprint, palm and full-hand scanners, voice, facial recognition systems, iris scanning technology, pupil scans, document readers, biometric software, and related services capable of wireless, mobile or stationary use to limit access to the patient or physician. The term also incorporates any system, while not biometric, that allows access via the use of a Login Name in combination with a Password and/or any additional security information, e.g., a computer-generated password that is sent by a server via email and/or text message, as well as programs developed to allow for the personalization of motions or movements, etc., to restrict access only to the patient or physician.

“Optional” or “optionally” means that the subsequently described element, component or circumstance may or may not occur, so that the description includes instances where the element, component, or circumstance occurs and instances where it does not.

“Patient Self-Assessment” as used herein means a range of potential types of measurements resulting from a (i) patient responding to a question, (ii) a self-administered test, (iii) a self-report input that is digitally captured, and/or (iv) digital diaries whose information can be quantified for use by the treating physician. Examples include, but are not limited to: (i) the level of pain (e.g., responding to the Mosby Pain Index, Wong-Baker Facial Grimace Scale, etc.), (ii) an activity tolerance scale, (iii) a quality of life scale, (iv) a discomfort scale, (v) a physiologic value (e.g., forced expiratory volume (FEV), blood pressure, heart rate, heart rate variability, eye dilation, balance, gait, weight, food consumption, Galvanic skin resistance, non-invasive CNS activity such as but not limited to cortical activity assessed via regional cerebral blood flow (rCBF), electroencephalogram (EEG), spectral EEG, event related potentials, and other possible physiological indices of CNS activation), (vi) stress, (vii) blood oxygen saturation (SpO2), etc., or a circulating compound in blood for more chronic disease state monitoring.

“Pharmacodynamics” means the biochemical and physiological effects of drugs on the body or on microorganisms or parasites within or on the body and the mechanisms of drug action and the relationship between drug concentration and effect.

“Pharmacokinetics” means the study of the bodily absorption, distribution, metabolism, and excretion of drugs.

The terms “subject” or “patient” are used interchangeably herein and refer to a human or other mammal.

The term “therapeutically-effective amount,” as used herein, refers to the amount of the biologically active agent needed to stimulate or initiate the desired beneficial result. The amount of the biologically active agent employed will be that amount necessary to deliver an amount of the biologically active agent needed to achieve the desired result. In practice, this will vary widely depending upon the particular biologically active agent being delivered, the site of delivery, and the dissolution and release kinetics for delivery of the biologically active agent into skin of the affected area and the patient's individual response to dosing.

### Devices

The present disclosure relates to devices useful for peripheral nerve stimulation in order to modulate the autonomic nervous system. Various devices may be employed, for example, the devices described in U.S. Provisional Patent Application Ser. No. 62/525,151 filed on Jun. 26, 2017, titled “Methods and Apparatus for Vagus Nerve Stimulation,” which is owned by Applicant and incorporated herein by reference in its entirety.

Aspects of the invention involve systems and method for delivery of an electrical signal to one or more target neural structures. In some embodiments, the target neural structure may be a vagus nerve structure. In one embodiment, the target neural structure may be a vagus nerve structure in the ear of a patient. In some embodiments, the signal may be a high frequency pulsed electrical signal.

Studies have shown that specific structures on the pinna of the ear have corresponding subcutaneous neural structures. Peuker et al., in “The Nerve Supply of the Human Auricle,” Clinical Anatomy, 15:35-37 (2002), established that the auricle or pinna of the human ear includes the auricular branch of the vagus nerve, the greater auricular nerve, and the auriculotemporal nerve. In addition, it was also shown that the auricular branch of the vagus nerve was present at the cymba concha (100% of subjects), antihelix (73% of subjects), tragus (45% of subjects), cavum concha (45% of subjects), the crus of helix (20% of subjects), and the crura of the antithelix (9% of subjects). Similar observations were made for structures associated with the greater auricular nerve and auriculotemporal nerve. Accordingly, in some embodiments of the invention, an electrical signal is applied to one or more of the foregoing structures.

Although the device of the present disclosure is specifically described with respect to particular nerves, different peripheral nerves or combination of nerves may be used as entrance points for multiple device variations in order to achieve the desired therapeutic effect. The peripheral nerve(s) to be used as the entrance point will depend upon the therapeutic area.

In addition, although the device of the present disclosure is specifically described with respect to delivering a particular means of stimulating the peripheral nerve, other means of stimulation may be used in addition to electrical stimulation, such as optical stimulation and mechanical stimulation.

In one embodiment, the control unit is able to influence the (a) frequency of an alternating current, (b) level of the current, (c) length of impulses of the current, (d) stimulation time intervals of the current, (e) time profile of the current flowing through the electrodes and/or (f) stimulation electrodes. A rechargeable battery is optionally arranged in the device and supplies current to the control unit.

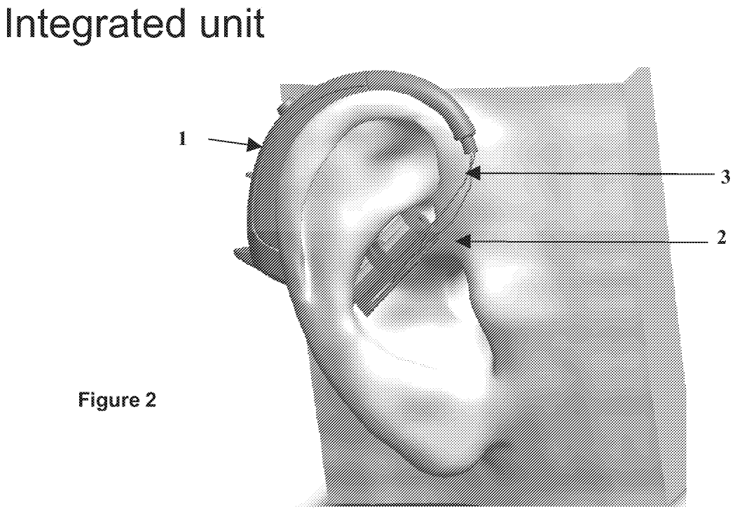

In yet another configuration, the integrated unit has a control unit (1), one or multiple electrodes (2) in conchae (potentially including the cymba) and or the ear canal, and wires (3) that connect the control unit with the electrode(s) as shown in FIG. 2. The electrodes can be completely self-sustaining (for example, a battery may be incorporated in the electrodes) and communicate wirelessly with the control unit.

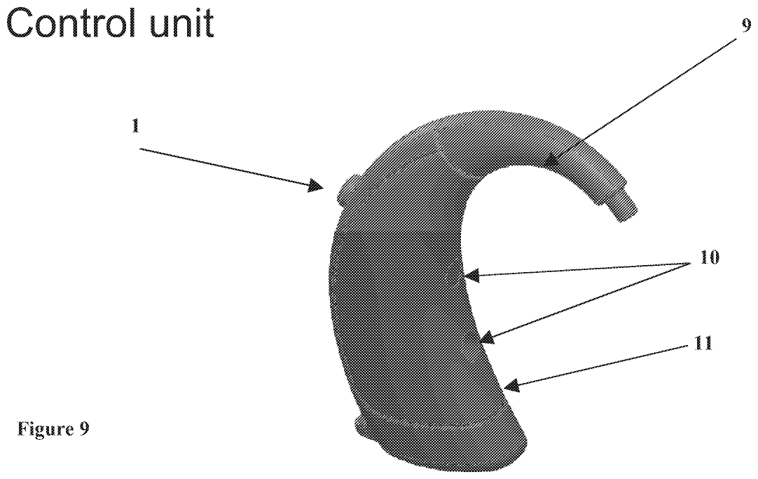

In another embodiment, the control unit (1) may have one or more of following attributes: houses stimulator, electrodes that stimulate the back of the ear, or a photoplethysmography (PPG) system to measure heart rate variability (HRV) as shown in FIGS. 2 and 9.

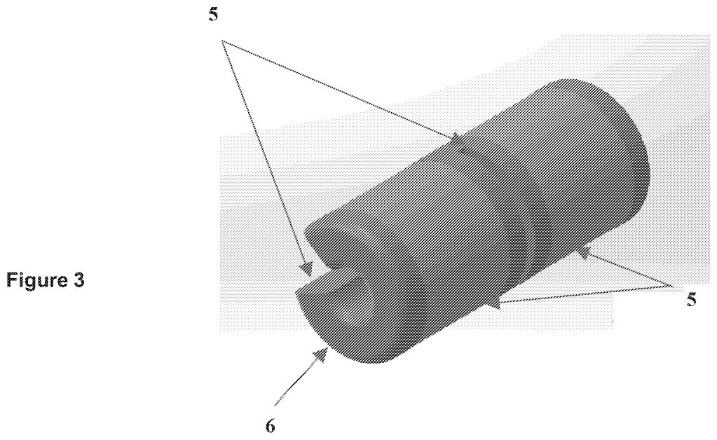

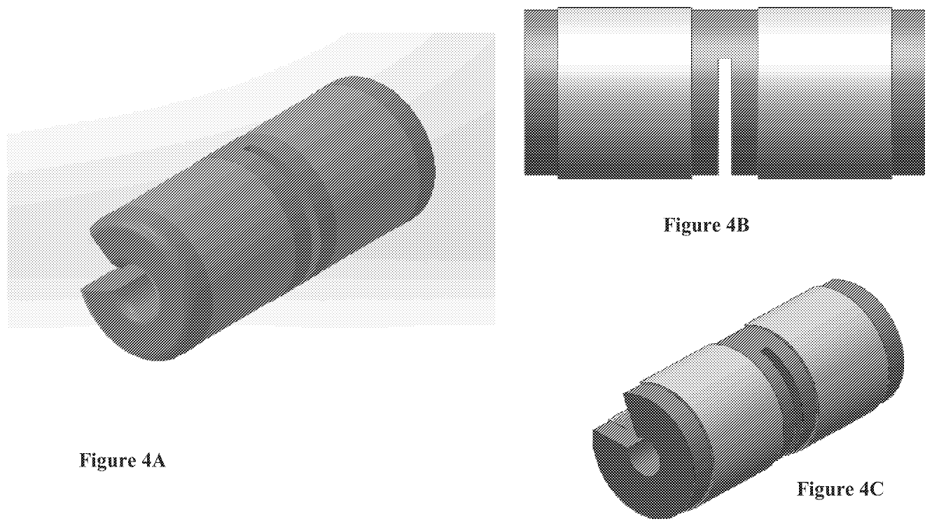

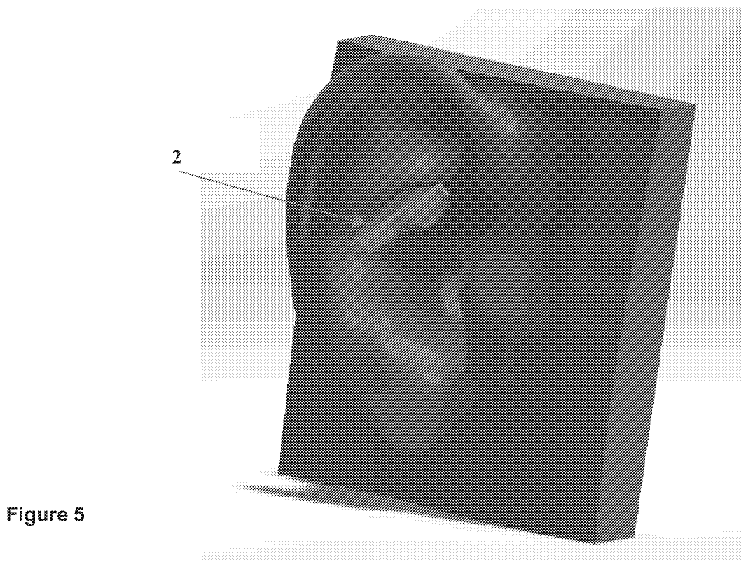

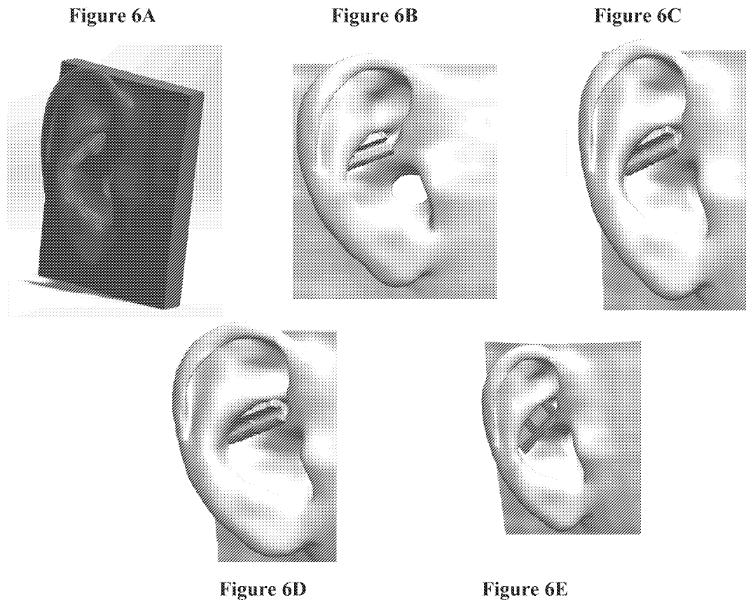

The electrode (2) is made out of a metal or a conductive plastic (4) and has cut-outs (5) to increase the flexibility of the electrode (FIGS. 3 and 4A-C). The electrode (2) may be of sufficient flexibility to comply with the cymbae conchae anatomy (FIGS. 5, 6A-E, and 8).

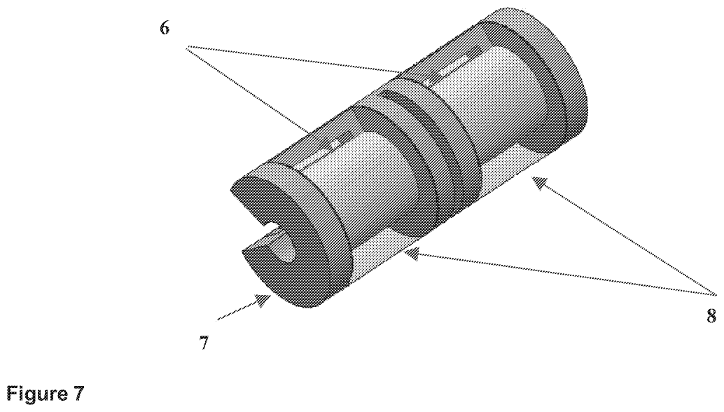

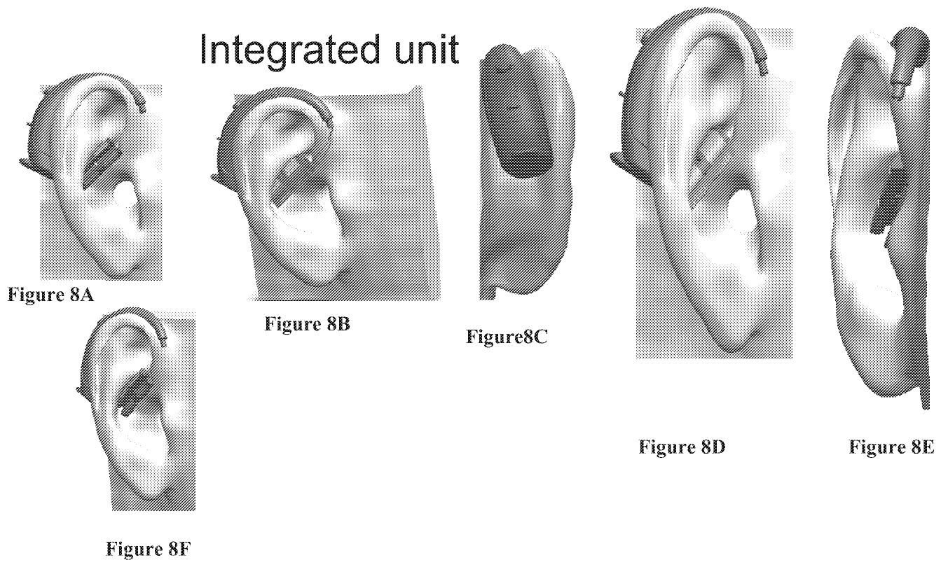

The electrode comprises: (i) a conductive sheet (6) that ensures the uniformity of the current, (ii) a base (7) that is made out of flexible material, and (iii) a conductive plastic coating (8) that allows the electrode to confirm with the anatomy of the ear (FIG. 7).

The device may further comprise a sensor or be linked to a sensor for measuring a physiological parameter of the patient. This parameter can, for example, be the patient's pulse or the oxygen saturation of the patient's blood or the FEV, blood pressure heart rate or heart rate variability or cortical regional blood flow. A memory chip can also be provided for storing the data measured by means of the sensor. The same or different sensor data or different analysis of the data might drive the stimulation parameters on the nerve or nerves being stimulated.

The electrodes or other physiological sensing technologies can be integrated into the earpiece, the head band, or into the headset of a hands-free mobile telephone unit, and the control unit can be integrated into a mobile telephone. Provision can be made for the connection between electrodes and the control unit to be established via a wireless radio connection, in particular via a Bluetooth connection, WiFi connection, or a WLAN connection.

It is also possible for the electrodes to be integrated into the headphones of a music playback system, and for the control unit to be integrated into the music playback system.

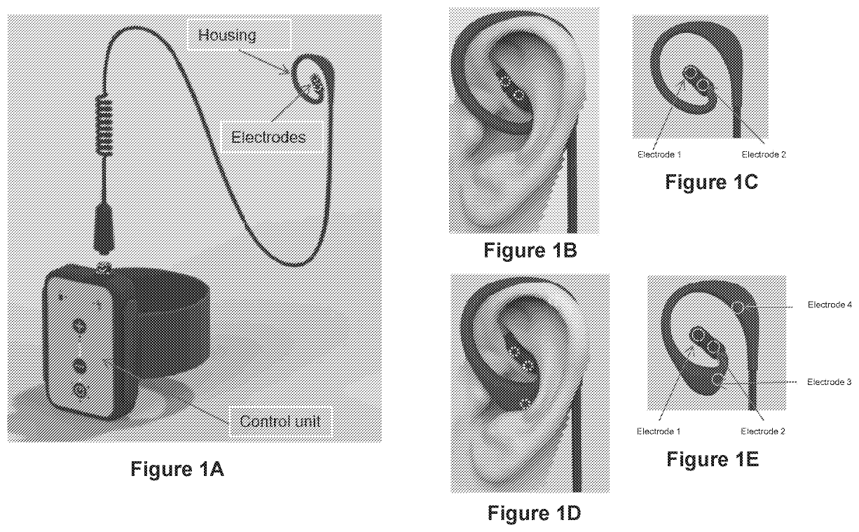

The present invention also relates to a method for the transdermal stimulation of a nerve of the human body, in particular of a part of the vagus nerve, by applying an electrical stimulus via at least one stimulation electrode and at least one reference electrode, at least one of which is placed in contact with the skin surface of concha and/or the ear canal of one or both of the human ears. The invention may also, via selective current delivery to other locations of the ear, modify the activity of the autonomic nervous system by affecting the parasympathetic and/or the sympathetic activity using a combination of electrodes as seen in FIG. 1.

By modulating the field vector and the frequency of the electrical stimulation, the present invention can potentially target both afferent and or efferent fibers on the vagus nerve. Also, by using one or both ears, the device may exploit the known difference in left versus right vagus nerves as principally an inflow or outflow system of the NTS, respectively. Afferent fibers, accessible in the tragal somatic representation of the vagus as well as sympathetic afferent neural inflows, will potentially enable the present invention to impact visceral sensory signal integration at higher CNS structures like the Nucleus Tractus Solitarius (NTS), RVLN, hypothalamus, and cortical structures related to autonomic function and Dorsal motor nucleus.

The present device and disclosure thus stimulates the peripheral nerves (e.g., the nerve branches (auricular branch) of the vagus nerve in the area of the external auditory canal) and thus influences CNS control of inflammation. This is achieved by integrating the technology of transdermal vagus nerve stimulation into a stimulation device, which is to be worn on or behind the ear and whose outward appearance is similar to that of a hearing aid or audio headset in other configurations.

When the earpiece is in use, the electrodes touch the skin surface area of external auditory canal “as well as” the auricle and are therefore able to modulate the autonomic system by selectively affecting both the sympathetic and/or parasympathetic side. Additional features include the following:
Range of stimulation frequency: about 1 Hz to 50 kHz.
Range of stimulation strength: about 0-10 mA.
Description of vectors: towards sympathetic and parasympathetic targets on the ear.
Duration of treatment: up to 1 hour at each session, and not more than twice daily is preferred.

In certain embodiments, the use of the device induces no feeling to patients and is devoid of unintended and unpleasant sensations, e.g., tingling, paresthesia, pain, hoarseness, voice impact, etc. A device that is comfortable is not only important to patient compliance but also to ensure the blinding in controlled clinical trials.

In a further alternative, the stimulation technology can be integrated into a mobile telephone and into its hands-free unit. The control unit and its electronics can in this case be integrated into the circuitry of the mobile telephone. The stimulation unit can be installed in the earpiece of the hands-free unit. The communication between earpiece and mobile telephone can be wireless, for example by means of Bluetooth technology, or can be via a connecting cable.

It is also possible for the technology to be integrated into headphones and devices, for example, for digital media playback. These devices can be, for example, MP3 players or iPods.

In another alternative, sensors will be integrated in the control unit and/or housing. Based on the sensor output, the control unit will automatically switch on/off the stimulation or change the stimulation parameters to provide effective therapy. The inventive devices have the ability to communicate with the sensors to optimize the particular therapy based on sensor readings. Such sensor measurements may comprise sleep quality, activity (based on accelerometer, gyroscope, Global Positioning Systems (GPS)), blood pressure, heart rate, heart rate variability, oxygen saturation and the like or indices of neural activation and modulation. Sensors may be integrated in the headphones (neural interface) or they may be standalone products which interact and communicate with the neural interface.

Another feature of the present invention is that the devices can be programmed to apply simultaneous or phased stimulation at different locations on the ear(s). Different therapeutic parameters (e.g., frequencies) may be employed and can be personalized for the patient based on the data received from the sensor about the patient's condition. Stimulation ramp-up can occur at startup. The programmable stimulator ramps up the final current over a period of time so that the patient does not feel any sensation associated with rapid current transition.

The present invention in certain embodiments also has the ability to provide therapeutically effective levels of nerve stimulation to peripheral points other than the ear(s) by using different nerves as the conduits to the brain. For example, other nerves such as the radial nerve, vagus nerve (around the neck), and trigeminal nerve may be targeted. In other aspects, the device can be designed and programmed to provide stimulation to both the ear stimulation points as well as other peripheral nerves.

The present invention in certain embodiments also has the ability to provide therapeutically effective levels of nerve stimulation using non-electrical stimulation to peripheral points other than the ear(s) by using different nerves as the conduits to the brain. For example, other nerves such as the optical nerve using different light wavelengths to stimulate.

In another embodiment, the components of the device are all contained in the ear, with features to securely and optimally place the electrodes using anatomical features.

In another embodiment, the device consists of features that are optimally designed to fit the device intuitively into the ear based on consistency of anatomical guide surfaces and angles across a multitude of ear geometries.

Another embodiment consists of a wearable gear such as but not limited to a headband or ear mitt to house the stimulation, sensing, and/or audio components.

In another embodiment, artificial intelligence techniques can be used to optimize the duration and selection of electrode combinations for effective therapy and power consumption, taking into consideration inputs from other data sources and sensors that the user may interface with.

In some embodiments the device may use as neural sensing electrodes, near infrared sensors, or capillary bed sensing technologies to develop useful physiological signals for device control in a feed-forward fashion. These sensing devices include transcutaneous electrodes, optical sensing technologies both passive and active, and/or infrared cortical monitoring techniques. These allow for acquisition of direct and indirect CNS activity and its response to autonomic neuromodulation as stated in the various claims, designs, and embodiments herein.

Other features of the inventive devices include: (a) the ability to modify therapeutic doses of stimulation through a software application (an “app”) for a mobile electronic device (such as an iPhone or an Android-based mobile device) based on clinician guidelines and patients' adherence to the app; (b) verbal response options to provide patients with verbal statements about status of therapy, feedback, or instructions; (c) the ability to modulate the maximum amplitude (or other parameters) of the therapy for the user based on their conditioning and/or other sensor responses; (d) a hub-and-satellite model of non-invasive stimulation with the headphone as a central unit and other “satellite beacons” at key points on the body (e.g., reaching the splenic, saphenous, or peroneal nerves); (e) synchronized therapy between the hub and satellites to modulate quantification of inflammatory signal from the peripheral organs, and subsequently the anti-inflammatory response; (f) optimization of sensor module construction and/or location(s) to minimize noise from therapy; (g) monitoring the count of the doses by the app or the hardware; (h) the ability for the patient to purchase a therapy session using the app or through some other companion device; (i) the ability for clinicians to monitor the patients' conditions and responses to therapy over the internet (Health Insurance Portability and Accountability Act of 1996 “HIPPA” compliant if indicated) and allowing clinicians to change the parameters of therapy via internet-enabled communications; and (j) the therapy apparatus is contained in the headphone ear pad.

Based on metrics received from the sensor data, the physician can, in the initial office visit, determine whether the patient has responded positively to the first treatment. The physician can adjust the level of stimulation and/or pharmacotherapy accordingly.

In one embodiment, one or more peripheral nerve is stimulated with implanted electrodes and no stimulation induced sensation. As an example an implantable electrode can be placed in the proximity of the radial or tibial nerve through a small incision. Electronics and battery can be buried under the skin or remote energy delivery can be used.

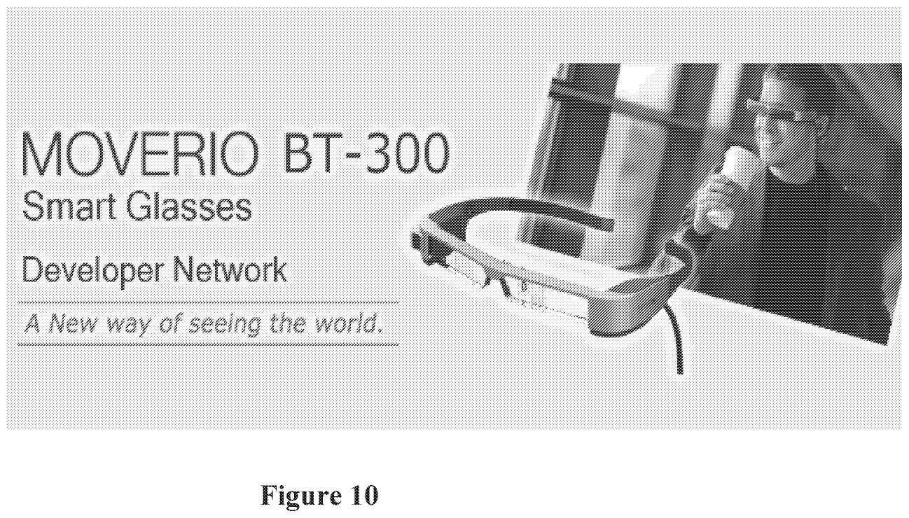

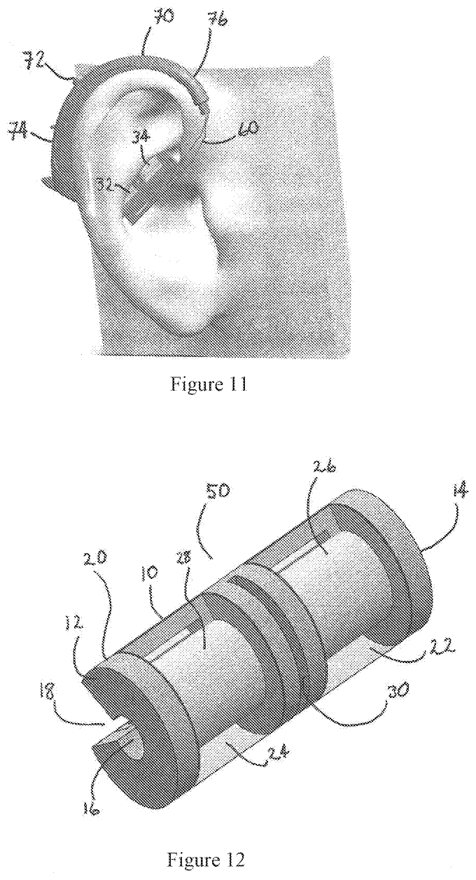

In another embodiment, the device may stimulate the optical nerve (using light waveforms as opposed to electrical stimulation) to restore gamma waves. Disorganized gamma waves are a predictor to Alzheimer's disease. Restoring normal gamma waves result in reduction in amyloid plaques in an Alzheimer's animal model. In order to stimulate the optical nerve, white light or specific wavelengths within the visible and non-visible spectrum is/are used. This optical stimulation can be used by itself or in combination with electrical stimulation of a peripheral nerve (e.g., the auricular vagus) or any other nerve (FIG. 10).

In some embodiments, an interface is provided with electrodes to engage a target area of the skin of the ear of a patient that is adjacent to a target subcutaneous neural structure, and stimulation of a target neural structure is delivered transcutaneously across the skin via the electrodes that engage the skin. In one embodiment, an electrical stimulation module applies a high frequency pulsed electrical signal to the neural structure. In some embodiments, a low frequency (or non-high frequency) pulsed electrical signal is applied. As defined herein, high frequency stimulation involves the delivery of a pulsed electrical signal at a pulse frequency exceeding 500 Hz. In various embodiments, pulse frequency ranges may comprise 1 Hz to 100 kHz, 1 Hz to 50 kHz, 1 kHz to 100 kHz, 3 kHz to 50 kHz, 5 kHz to 50 kHz, 10 kHz to 40 kHz, 10 kHz to 25 kHz, 15 kHz to 25 kHz, and about 20 kHz. In some embodiments, application of a high frequency pulsed electrical signal capable of generating afferent or efferent action potentials in a vagus nerve structure is provided. In some embodiment, a pulsed electrical signal is generated by an electrical stimulation module and delivered by one or more electrodes coupled to a generally cylindrical interface member having a C-shaped cross-section adapted to engage a target portion of the skin of an ear of the patient. In some alternative embodiments, the interface member may comprise a generally cylindrical member that is not C-shaped in cross-section. In other alternative embodiments, the interface may comprise a member that has a C-shaped cross-section but is not cylindrical.

In some embodiments, a neurostimulation therapy is provided to a neural structure in the ear of a patient by applying a high frequency pulsed electrical signal to the skin of a target portion of the ear that is proximate to the neural structure. In some embodiments, the high frequency pulsed electrical signal reduces at least one proinflammatory biomarker and increases at least one anti-inflammatory biomarker. In some embodiments, a first high frequency pulsed electrical signal is applied to the skin adjacent to a first neural structure in the ear of a patient, and a second high frequency pulsed electrical signal is applied to the skin adjacent to a second neural structure in the ear of the patient, and each of the first and second high frequency pulsed electrical signals produces a physiological effect selected from an increase in the patient's parasympathetic tone, a decrease in the patient's sympathetic tone, an increase in at least one anti-inflammatory biomarker, and a decrease in at least one pro-inflammatory biomarker. In some embodiments, application of a first high frequency electrical signal to a first vagus nerve structure and a second high frequency electrical signal to a second vagus nerve structure is provided, and the first and second electrical signals each produce a physiological effect selected from an increase in the patient's parasympathetic tone, a decrease in the patient's sympathetic tone, an increase in at least one anti-inflammatory biomarker, and a decrease in at least one pro-inflammatory biomarker.

Certain embodiments may be understood in connection with the Figures in which like numbers are referred to like elements throughout. FIG. 11 illustrates one embodiment of an electrical neurostimulation system for providing an electrical neurostimulation signal to a target portion of an ear of a patient. The system includes an interface member (50) sized and shaped to engage the target portion of the ear. In the embodiment of FIG. 11, the interface member 50 is adapted to engage and fit securely within a cymba concha of an ear of a patient. The interface includes an electrode pair 32, 34 for delivering the electrical neurostimulation system to a vagus nerve structure adjacent to the cymba concha. In alternative embodiments (not shown) one or more electrodes may be coupled (e.g., by wire or wirelessly) to electrodes placed on the skin adjacent to alternative or additional target portions of the patient's ear (e.g., an antihelix, a tragus, an antitragus, a cavum concha, a helix, a scapha, a triangular fossa, or a lobule) to stimulate a neural structure selected from a vagus nerve structure, a greater auricular nerve structure, and an auriculotemporal nerve structure.

As shown in FIGS. 11 and 16, an electrical stimulation module 70 is coupled by lead wires 60 to the electrodes 32, 34. In alternative embodiments (not shown) the electrical stimulation module 70 may be wirelessly coupled to the electrodes 32, 34 via RF energy. In a still further alternative to the embodiment of FIG. 11, the electrical stimulation module may be miniaturized and located entirely on or within the interface 50, such that the interface, electrode(s) and stimulation module comprise an integrated system.

The electrical stimulation module 70 may include a processor and other circuitry to generate and control the delivery of an electrical signal to the electrodes 30, 32. In one embodiment, a processor includes a pulse generator and a controller to generate and deliver to the electrodes 30, 32 electrical pulses according to one or more parameters (e.g., pulse frequency, pulse width, current amplitude, voltage amplitude, ON time, OFF time, therapy delivery time, etc.) defining the electrical signal. The electrical stimulation module 70 may also include additional circuitry elements, e.g., logic gates, clocks, voltage and current sources, D/A converters, comparators, output circuits, etc., useful or necessary to generate and deliver the electrical signal. A programmer (not shown) may be used to wirelessly program the electrical stimulation module 70.

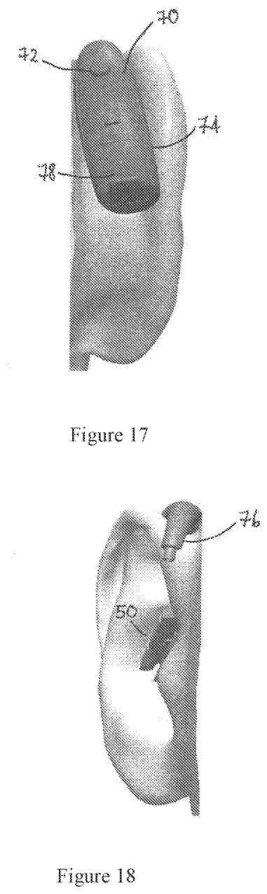

As shown in FIGS. 11, 16, 17, and 18, electrical stimulation module 70 includes a generally curved body adapted to fit behind the ear (i.e., between a lateral surface of the ear and the skin overlying the skull (see FIGS. 17, 18). An upper portion 76 is adapted to curve over the ear toward the patient's face as shown in FIG. 18, which is a front view of a right ear of the patient. A lower portion is located posteriorly behind the ear, as shown in FIG. 17, which is a rear view behind the patient's right ear. The electrical stimulation module preferably includes a power supply (e.g., a battery), and maintained in the electrical stimulation module 70 by a power supply cover 78. An on/off button 72 is also provided to enable a patient to manually turn the unit on or off.

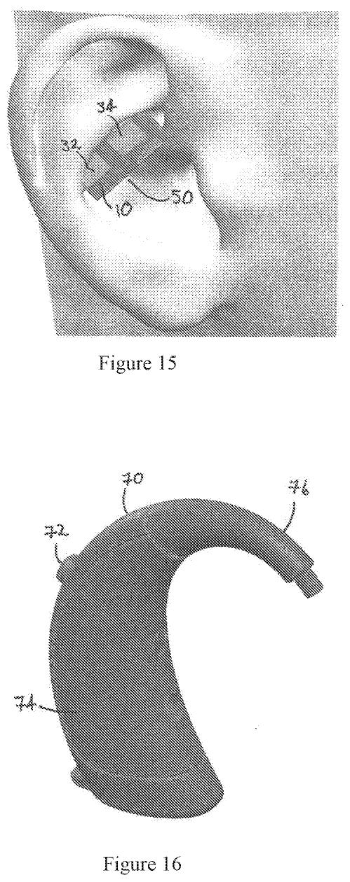

FIG. 12 illustrates a frame 10 and a first interface member 50. First interface member 50 is adapted to engage and fit securely in place at a target location on the patient's ear, as shown in FIG. 15. Frame 10 of FIG. 12 includes a generally cylindrical body having first and second lateral ends 12, 14 of the generally cylindrical body. Frame 10 is C-shaped, as defined by an open portion 18 of the generally cylindrical body and a bore 16 passing axially through the body of the cylinder. An external periphery 20 includes first and second cutout or notched areas 22, 24, extending between cylindrical cores 26 and 28. In one embodiment, the frame 10 is comprised of one or more resilient polymers, e.g., silicone-based polymers, and the patient may compress the C-shaped frame 10 to enable the first interface member 50 to be easily fitted within a target area of the ear such as the cymba concha, as shown in FIG. 15.

In one aspect, embodiments of the present disclosure include electrical stimulation systems for providing a neurostimulation signal to a target portion of an ear of a patient. In one embodiment, an interface member is provided to engage the target portion of the ear. The interface member may be sized and shaped to conform to the anatomy of the target portion. In some embodiments, the interface member is a resilient member that may be compressed or otherwise temporarily deformed by the user to engage the target portion of the ear and, after being placed adjacent to the target portion, retained in place by the natural anatomy. One such embodiment is illustrated in FIG. 14, which depicts a generally cylindrical, flexible interface member having a C-shaped cross-section being retained in place by a compressive or frictional fit within the cymba concha. Other interfaces may similarly engage other anatomical sites. In alternative embodiments, similar systems may be shaped to engage neural structures adjacent to other target areas of the body.

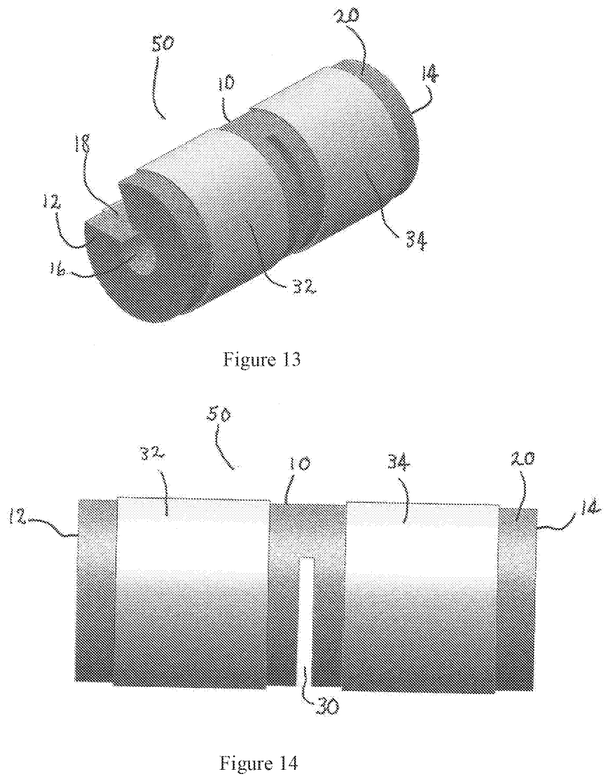

In one embodiment, the external periphery of the interface member includes at least one electrode coupled to or integrally formed thereon. The electrode may comprise an electrode pair (i.e., a cathode and an anode) in some embodiments. When the interface member is retained adjacent to the target area, the electrode is adapted to contact the skin of the target portion of the ear (which may be a left ear or a right ear). The electrodes may comprise any number of suitable materials, including metals such as stainless steel, platinum, platinum-iridium alloys, and conductive polymers such as carbon-loaded silicone. The electrode delivers the first electrical signal transcutaneously to a neural structure proximate the target portion of the ear, such as a vagal structure adjacent the cymba concha (FIG. 14). The electrode may be sized to provide a current flux capable of inducing action potentials on one or more nerve fibers of the neural structure. As shown in FIGS. 13 and 14, an electrode pair 32, 34 on the outer periphery 20 of the first interface member 50 may deliver the electrical signal. Target portions of the ear may include, without limitation, an antihelix, a tragus, an antitragus, a cavum concha, a helix, a scapha, a triangular fossa, a lobule, and a lateral surface (i.e., backside surface of the ear facing the skull of the patient). Adjacent neural structures may include a vagus nerve structure, a greater auricular nerve structure, and an auriculotemporal nerve structure.

In one embodiment, at least one electrical stimulation module is coupled to the at least one electrode, and is capable of generating and applying a first electrical signal to the electrode(s). In one embodiment, the first electrical signal is a pulsed electrical signal defined by a plurality of parameters. The parameters may include a pulse frequency, a pulse width, and a current amplitude. In alternative embodiments, an ON time (during which the pulsed electrical signal is delivered as a programmed frequency and current amplitude is applied to the nerve), and an OFF time (during which no signal is applied to the nerve) are also among the parameters defining the first electrical signal. Pulse frequencies for the first electrical signal may range from 5 Hz to 50 kHz.

In one embodiment, the first electrical signal is a high frequency signal having a frequency range, in various embodiments, from 1 kHz to 100 kHz, 3 kHz to 50 kHz, 5 kHz to 50 kHz, 10 kHz to 40 kHz, 10 kHz to 25 kHz, 15 kHz to 25 kHz, and about 20 kHz. Although it is widely believed that neurostimulation, and particularly vagus nerve stimulation, at frequencies above 500 Hz preclude generation of action potentials in the neural structure, applicants have discovered that stimulation above frequencies of 1 kHz can have desirable physiological effects, which may include, without limitation, an increase in one or more anti-inflammatory biomarkers, a decrease on one or more proinflammatory biomarkers, an increase in the patient's parasympathetic tone, and a decrease in the patient's sympathetic tone.

Current amplitudes in the first electrical signal may range from 0.1-20 milliamperes (mA). Pulse widths in the electrical signal may range from 1-500 microseconds, 10-50 microseconds, and 10-30 microseconds in various embodiments. In a particular embodiment, the electrical signal may have a pulse frequency of 10 kHz to 25 kHz, a pulse width of 10-30 microseconds, and a current amplitude of at least 5 mA.

In different embodiments, the at least one electrode may be coupled to the electrical stimulation module by wire or wirelessly. In the embodiment of FIG. 11, the electrodes 32, 34 are coupled to the electrical stimulation module 70 by a lead wire 60.

In one embodiment, the electrical stimulation system includes a second interface member, at least one second electrode, and a second electrical stimulation module, which may be substantially similar to the first interface member, the at least one first electrode, and the at least one electrical stimulation module, respectively. The second interface, second electrode, and second electrical stimulation module (not shown) may be used to provide a second electrical stimulation signal to an opposite ear from that of the first electrical signal to provide a bilateral neural stimulation therapy to both sides of the patient's body. In one embodiment, the first and second electrical stimulation modules each include a transceiver, which are used to wirelessly couple the first and second electrical signal modules. The transceivers may allow the first and second electrical stimulation modules to synchronize the delivery of the pulses of the first and second electrical signals.

In alternative embodiments, the electrical stimulation system includes a feedback system for adjusting the delivery of the electrical signals to the target body structure. In one embodiment, the electrical stimulation system includes at least one sensor capable of sensing a body signal. The sensor may be selected from, without limitation, a cardiac sensor, a blood oxygenation sensor, a cardiorespiratory sensor, a respiratory sensor, and a temperature sensor. The system may also include a processor for determining a body parameter based on the body signal. For example, the processor may calculate a heart rate, heart rate variability, parasympathetic tone, sympathetic tone, or sympathetic-parasympathetic balance from a cardiac signal; a pulse oximetry value from a blood oxygenation signal; a breathing rate or end tidal volume from a respiratory signal; or an exertional level from an accelerometer coupled to the patient's body, etc. The electrical stimulation module may use the body parameter to adjust one or more parameters defining the electrical signal, e.g., the signal may be turned off if the patient's heart rate falls below a predetermined lower limit or if activity levels become elevated or depressed. In one embodiment, the sensor may be located on the skin of a lateral surface of the ear (i.e., the side of the ear facing toward the patient). In one embodiment, the sensor may be externally located on the skin of the patient's head below a mastoid. In a specific embodiment, the sensor on the lateral portion of the ear, or on the head, may be a cardiac sensor.

It is widely known that vagus nerve stimulation systems affect inflammatory biomarkers. Without being bound by theory, applicants believe that, according to one or more embodiments of the invention, an electrical stimulation system can provide a high frequency pulsed electrical signal to stimulate a vagus nerve structure in the patient's ear so as to reduce at least one pro-inflammatory biomarker and increase at least one anti-inflammatory biomarker.

In some embodiments, two electrical signals may be applied to different neural structures adjacent to two target portions of the ear of the patient, and each of the first and second electrical signals may provide a different physiological effect selected from an increase in one or more anti-inflammatory biomarkers, a decrease on one or more pro-inflammatory biomarkers, an increase in the patient's parasympathetic tone, and a decrease in the patient's sympathetic tone.

In another embodiment, two electrical signals are applied to two different anatomical sites of the patient, and each of the first and second electrical signals may provide a different physiological effect selected from an increase in one or more anti-inflammatory biomarkers, a decrease on one or more pro-inflammatory biomarkers, an increase in the patient's parasympathetic tone, and a decrease in the patient's sympathetic tone. For example, the anatomical sites are one wrist and one ear of the patient. In one embodiment, the two electrical signals are applied simultaneously. In another embodiment, the two electrical signals are applied consecutively.

In yet another embodiment, two or more electrical signals are applied to two or more different anatomical sites of the patient, and each of the electrical signals may provide a different physiological effect selected from an increase in one or more anti-inflammatory biomarkers, a decrease on one or more pro-inflammatory biomarkers, an increase in the patient's parasympathetic tone, and a decrease in the patient's sympathetic tone. In one embodiment, the two or more electrical signals are applied simultaneously. In another embodiment, the two or more electrical signals are applied consecutively.

Methods of Treatment

The present invention relates to methods of treating a disease or condition by modulating the autonomic nervous system response by affecting the sympathetic (SYMP) and/or parasympathetic (PSYMP) systems either alone or in combination with a therapeutic agent. Such diseases include but not limited to asthma, allergic rhinitis, Alzheimer's, autoimmune diseases, rheumatoid arthritis, inflammation, systemic lupus erythematosus, inflammatory bowel disease (IBD), ulcerative colitis, Crohn's disease, multiple sclerosis, diabetes, Guillain-Barre syndrome, chronic inflammatory demyelinating polyneuropathy, psoriasis, thyroid disorders, myasthenia gravis, and vasculitis. More particularly, therapeutic uses of the present invention comprise treating hypertension (particularly uncontrolled hypertension), inflammation after stroke, myocardial infarction recovery, anesthesia-induced inflammatory response, influenza, atrial fibrillation, relapse from cardio-conversion, sepsis, ventricular and supraventricular arrhythmias, autoimmune-mediated glomerulonephritis, Berger's IgA nephropathy, demyelination syndromes (e.g., multiple sclerosis, Devic's syndrome etc.), severe allergic reactions (e.g., skin, lungs), and autoimmune diseases (e.g., pancreatitis, gastritis, thyroiditis, hemolytic anemia, encephalitis, myasthenia gravis).

In one embodiment, the devices and methods of the present invention can be configured in an in-office trial where the clinician will assess the effect of the treatment on a physiologic parameter, e.g., heart rate variability to find the optimal electrodes, frequency etc. Or the device could be provided with the optimal parameters for the disease stage of the patient based on the clinical trial data. In such case, if the patient is adequately responding, then the patient may be provided with the device and instructions for home use.

The therapeutic agents useful in the inventive methods include, but are not limited to, abatacept, adalimumab (Humira®), adalimumab-atto, anakinra, certolizumab, etanercept, etanercept-szzs, golimumab, infliximab, infliximab-dyyb, rituximab, tocilizumab, tofacitinib, methotrexate, and an NSAID. New agents presently in clinical trials are also contemplated by this disclosure.

Agents useful in the treatment of asthma include inhaled corticosteroids, leukotriene modifiers, long-acting beta agonists (LABAs), theophylline, short-acting beta agonists such as albuterol, ipratropium (Atrovent®), intravenous corticosteroids (for serious asthma attacks), allergy shots (immunotherapy), and omalizumab (Xolair®).

In some embodiments, the combination therapy of autonomic system modulation by affecting both the sympathetic (SYMP) and/or parasympathirc (PSYMP) activation and a therapeutic agent can result in decreasing the dose needed for effectiveness of about 10%, or 15%, or 20%, or 25%, or 30%, or 35%, or 40%, or 45%, or 50%, or 55% or 60% and may also, via dose reduction, reduce untoward side effects.

In some embodiments, the combination therapy of autonomic modulation and a therapeutic agent can result in improving the response rate of the agent by 10%, 20%, 30%, 50%, or 75%, i.e., improve the percentage of patients that respond to the therapy based on the clinical definition of response using validated measure(s). The present invention could also increase the duration of time the biologic is effective before the patient becomes unresponsive to the drug. In other embodiments, the present invention may be used in combination with lesser expensive therapeutic agents such as methotrexate. Such combination is expected to improve the efficacy of the methotrexate (or other agent) so as to obviate the need for a more expensive biologic agent and/or prolongs the time before which the patient requires the biologic agent.

The device of the present disclosure has the ability to stimulate nerves without adverse effects or uncomfortable sensations. This is due, in part, to the use of higher frequencies (likely above about 5 kHz). Further, there may be a reduction in the infection rate (for rheumatoid arthritis) and reduced use of steroids (in asthma) as well as other potential unwanted side effects of pharmaceutical intervention. The present invention also has the ability to up-regulate and down-regulate afferent and/or efferent neural traffic. This is accomplished by targeting a field of afferent fibers to either the left or right vagus via the NTS. As an example, the left vagus has mostly afferent fibers and therefore stimulation of its cutaneous auricular afferents should inject signals into the left NTS, changing the neural integration therein. Likewise, stimulation of the right auricular vagal cutaneous somatotopy will influence the right NTS, whose principal outflow, the right vagal nerve, is principally efferent. Both left and right tragus have, outside of the vagal somatotopic representation, cutaneous nerve afferents that influence the sympathetic nervous system via the RVLN.

The present invention further provides the ability to modulate the autonomic nervous system by selectively up- or down-regulating sympathetic and parasympathetic activity by targeting specific stimulation locations that trigger sympathetic or parasympathetic effect (see above). The patient may place the wearable device on the stimulation points (as an example on or in one or both the ears) and turns on the stimulation. The sessions may be daily or on an as-needed basis.

In one embodiment, the duration of the session is about 10 minutes, about 15 minutes, about 20 minutes, about 25 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, or about 1 hour.

In another embodiment, the present invention is useful in treating hypertension. In one embodiment, static or dynamic kHz frequency (e.g., 5 kHz, 10 kHz, 15 kHz, 20 kHz, 25 kHz, or 50 kHz) stimulation to impact either or both SYMP and PSYMP activity at one or multiple peripheral nerves, combinations with other peripheral nerve targets may help achieve clinical satisfactory blood pressure control in conjunction with or without pharmacological interventions. Effects may include lowering of systolic and/or diastolic blood pressure, concomitant MAP, and or compensatory heart rate responses. An 5-15% reduction in systolic blood pressure and similar effect on heart rate could be possible. A lag time (time to get back to pre-stimulation baseline) comparable to the stimulation duration is possible although a much longer lag time is possible following a single of multiple stimulation sessions. A relationship between lag time and cumulative stimulation time delivered might be observed.

In one aspect, embodiments of the present disclosure include methods for providing a neurostimulation therapy to a neural structure in the ear of a patient. In one embodiment, the method comprises generating a high frequency pulsed electrical signal, and applying the signal to the skin of a target portion of the ear of the patient, proximate to a neural structure in the ear of the patient. The high frequency pulsed electrical signal may have a frequency, in various embodiments, ranging from 1 kHz to 100 kHz, 1 kHz to 50 kHz, 3 kHz to 50 kHz, 5 kHz to 50 kHz, 10 kHz to 40 kHz, 10 kHz to 25 kHz, 15 kHz to 25 kHz, and about 20 kHz. As previously discussed, it is widely believed that neurostimulation (e.g., vagus nerve stimulation) at frequencies above 500 Hz preclude generation of action potentials in the neural structure. However, applicants have discovered that stimulation above frequencies of 1 kHz can have desirable physiological effects including, without limitation, an increase in one or more anti-inflammatory biomarkers, a decrease on one or more pro-inflammatory biomarkers, an increase in the patient's parasympathetic tone, and a decrease in the patient's sympathetic tone.

In one embodiment, the high frequency pulse width may be defined by a pulse width and a current magnitude. The electrical signal may be provided, in various embodiments, with pulse widths of from 1-500 microseconds, 1-250 microseconds, 1-100 microseconds, 5-50 microseconds, 10-50 microseconds, and 10-30 microseconds. The electrical signal may be provided with current magnitudes of from 0.1-20 mA, 1-20 mA, and 5-15 mA in various embodiments.

In some embodiments, the high frequency pulsed electrical signal may be defined by additional parameters including an ON time, an OFF time, and a therapy delivery time. The ON time may comprise a time within a range of from 1 second to 12 hours, 5 seconds to 180 minutes, 5 seconds to 1 minute, and 5-30 seconds in various embodiments. The OFF time may comprise, in various embodiments, 1 second to 1 month, 5 seconds to 1 day, 5 seconds to 180 minutes, 5 seconds to 60 minutes, and 5 seconds to 10 minutes. The therapy delivery time may comprise a time of from 5 minutes to one month, 5 minutes to 24 hours, 1-24 hours, 3-12 hours, or 3-6 hours in various embodiments. The therapy delivery time may also begin at a programmed time of day comprising the foregoing time periods.

In one embodiment, the method may comprise providing an interface member having an external periphery comprising at least one electrode (e.g., an electrode pair 32, 34 as shown in FIGS. 13-15) and contacting the skin of the target portion of the ear with the at least one electrode. In one embodiment, the interface member may be provided having a generally cylindrical shape, and may comprise a resilient polymer. In a particular embodiment (as shown in FIGS. 13-15), the generally cylindrical interface member may be provided having a C-shaped cross-section, with the at least one electrode (e.g., electrode pair 32, 34) on an external periphery of the interface member.

The method may also comprise providing an electrical signal module, coupled to the electrode(s), generating the high frequency pulsed electrical signal using the electrical signal module, and applying the electrical signal to the skin of the target portion of the ear using the electrode(s). Providing the electrical signal module may including providing an electrical signal module that is coupled to the electrodes in various ways. In one embodiment, the electrical signal module may be wirelessly coupled to the electrode(s), while in an alternative embodiment, the electrical signal module is coupled to the electrode(s) by one or more lead wires such as lead wires 60 in FIG. 11. In a particular embodiment, the method comprises providing a miniaturized electrical signal module that is part of the interface member, and is coupled to the one or more electrodes by direct connection or by lead wires.

In one embodiment, the method may comprise contacting the electrode to the skin of a target portion of the ear such as the cymba concha, an antihelix, a tragus, an antitragus, a cavum concha, a helix, a scapha, a triangular fossa, a lobule, and a lateral surface of the ear (i.e., the side of the ear facing the patient), and applying the electrical signal to a neural structure proximate the target portion. The method may comprise applying the transcutaneously via the skin of the target portion to a neural structure selected from a vagus nerve structure, a greater auricular nerve structure, or an auriculotemporal nerve structure.

Some embodiments of the method may include adjusting one or more parameters defining the pulsed electrical signal based on feedback from the patient's body or, in some embodiments, the patient's environment (e.g., temperature, humidity, or time of day). In one embodiment, the method includes sensing at least one body signal of the patient, determining a body parameter based on the at least one body sensor, and adjusting the delivery of the electrical signal based on the body parameter. The method may comprise sensing one or more body parameters selected from a cardiac signal, a blood oxygenation signal, a cardiorespiratory signal, a respiratory signal, a temperature signal, and other body signals.

The method may also include providing a processor for determining a body parameter based on the body signal. For example, the processor may determine a heart rate, heart rate variability, parasympathetic tone, sympathetic tone, or sympathetic-parasympathetic balance from a cardiac signal; a pulse oximetry value from a blood oxygenation signal; a breathing rate or end tidal volume from a respiratory signal; an exertional level from an accelerometer coupled to the patient's body, etc. In one embodiment, one or more of the parameters defining the electrical signal (e.g., pulse frequency, pulse width, current amplitude, ON time, OFF time, or therapy delivery period) may be adjusted based on the value of the body parameter. The adjustment to the electrical signal parameter(s) may be performed by the electrical stimulation module based on logic circuitry, e.g. pulse frequency of the electrical signal may be increased or decreased if the patient's heart moves above or below predetermined limits, or if activity levels become elevated or depressed. In one embodiment, the sensor may be located on the skin of a lateral surface of the ear (i.e., the side of the ear facing toward the patient). In one embodiment, the sensor may be externally located on the skin of the patient's head below a mastoid. In a specific embodiment, the sensor on the lateral portion of the ear, or on the head, may be a cardiac sensor.

In one aspect, embodiments of the present disclosure include methods for providing a neurostimulation therapy to a neural structure in the ear of a patient. In one embodiment, the method comprises generating a pulsed electrical signal, and applying the signal to the skin of a target portion of the ear of the patient, proximate to a neural structure in the ear of the patient, so as to reduce at least one pro-inflammatory biomarker and increase at least one anti-inflammatory biomarker. The pulsed electrical signal may have a frequency, in various embodiments, ranging from 1 Hz to 100 kHz, 1 Hz to 50 kHz, 1 kHz to 100 kHz, 3 kHz to 50 kHz, 5 kHz to 50 kHz, 10 kHz to 40 kHz, 10 kHz to 25 kHz, 15 kHz to 25 kHz, and about 20 kHz.

Applicants have discovered that the electrical signal can be defined and applied so as to have desirable physiological effects including, without limitation, an increase in one or more anti-inflammatory biomarkers, a decrease on one or more pro-inflammatory biomarkers, an increase in the patient's parasympathetic tone, and a decrease in the patient's sympathetic tone.

In one embodiment, the method may comprise applying an electrical signal so as to reduce at least one pro-inflammatory biomarker selected from IL-1, IL-6, IL-12, IL-17, IL-18, C-reactive protein, TNF-α, INF-y, and increase at least one anti-inflammatory biomarker selected from IL-4, IL-10, IL-13, IFN-α, and TGF-8. In some embodiments, the method comprises applying the electrical signal so as to both reduce at least one of the foregoing pro-inflammatory biomarkers and increase at least one of the foregoing anti-inflammatory biomarkers.

In one aspect, embodiments of the present disclosure include methods for providing a neurostimulation therapy to a plurality of neural structures in a patient. The neurostimulation therapy comprises applying a first high frequency pulsed electrical signal to a first neural structure of a patient and a second high frequency pulsed electrical signal to a second neural structure the patient, with each of the first and second high frequency pulsed electrical signals having at least one physiological effect selected from an increase in the patient's parasympathetic tone, a decrease in the patient's sympathetic tone, an increase in at least one anti-inflammatory biomarker, and a decrease in at least one pro-inflammatory biomarker, with the effect of the first and second electrical signals being different.

The high frequency pulsed electrical signal may have a frequency, in various embodiments, ranging from 1 kHz to 100 kHz, 3 kHz to 50 kHz, 5 kHz to 50 kHz, 10 kHz to 40 kHz, 10 kHz to 25 kHz, 15 kHz to 25 kHz, and about 20 kHz.

In one embodiment, the high frequency pulse width may be defined by a pulse width and a current magnitude. The electrical signal may be provided, in various embodiments, with pulse widths of from 1-500 microseconds, 1-250 microseconds, 1-100 microseconds, 5-50 microseconds, 10-50 microseconds, and 10-30 microseconds. The electrical signal may be provided with current magnitudes of from 0.1-20 mA, 1-20 mA, and 5-15 mA in various embodiments.

In some embodiments, the high frequency pulsed electrical signal may be defined by additional parameters including an ON time, an OFF time, and a therapy delivery time. The ON time may comprise a time within a range of from 1 second to 12 hours, 5 seconds to 180 minutes, 5 seconds to 1 minute, and 5-30 seconds in various embodiments. The OFF time may, in various embodiments, 1 second to 1 month, 5 seconds to 1 day, 5 seconds to 180 minutes, 5 seconds to 60 minutes, and 5 seconds to 10 minutes. The therapy delivery time may comprise a time of from 5 minutes to one month, 5 minutes to 24 hours, 1-24 hours, 3-12 hours, or 3-6 hours in various embodiments. The therapy delivery time may also begin at a programmed time of day comprising the foregoing time periods.

In one embodiment, the method may comprise providing an interface member having an external periphery comprising at least one electrode (e.g., an electrode pair 32, 34 as shown in FIGS. 13-15) and contacting the skin of the target portion of the ear with the at least one electrode. In one embodiment, the interface member may be provided having a generally cylindrical shape, and may comprise a resilient polymer. In a particular embodiment (as shown in FIGS. 13-15), the generally cylindrical interface member may be provided having a C-shaped cross-section, with the at least one electrode (e.g., electrode pair 32, 34) on an external periphery of the interface member.

In one embodiment, the method may comprise contacting the electrode to the skin of a target portion of the ear such as the cymba concha, an antihelix, a tragus, an antitragus, a cavum concha, a helix, a scapha, a triangular fossa, a lobule, and a lateral surface of the ear (i.e., the side of the ear facing the patient), and applying the electrical signal to a neural structure proximate the target portion. The method may comprise applying the transcutaneously via the skin of the target portion to a neural structure selected from a vagus nerve structure, a greater auricular nerve structure, or an auriculotemporal nerve structure.

Some embodiments of the method may include adjusting one or more parameters defining the pulsed electrical signal based on feedback from the patient's body or, in some embodiments, the patient's environment (e.g., temperature, humidity, or time of day). In one embodiment, the method includes sensing at least one body signal of the patient, determining a body parameter based on the at least one body sensor, and adjusting the delivery of the electrical signal based on the body parameter. The method may comprise sensing one or more body parameters selected from a cardiac signal, a blood oxygenation signal, a cardiorespiratory signal, a respiratory signal, a temperature signal, and other body signals.

### EXAMPLES

The following examples are included to demonstrate certain embodiments of the present disclosure. Those of skill in the art should, however, in light of the present disclosure, appreciate that modifications can be made in the specific embodiments that are disclosed and still obtain a like or similar result without departing from the spirit and scope of the invention. Therefore, all matter set forth is to be interpreted as illustrative and not in a limiting sense.

### Example 1—Treatment of Rheumatoid Arthritis (RA)

A patient with poor RA control due to unacceptable side effects from immunosuppressive or biological therapies and a narrow therapeutic window (thresholds between efficacy and toxicity) may benefit from application of the invention to either raise the threshold effect for toxicity or lower the threshold effect for clinical benefit.

A patient with poor RA control due to inability to adhere to the complex medical regimes may find the invention easier to use and therefore achieve better RA control via improved compliance. A patient with poor RA control due to the financial burden of pharmaceutical interventions may have better control due to the one-time cost structure of the invention.

### Example 2—Treatment of Asthma

An asthmatic may have improved control due to an opening of the therapeutic window for pharmacological treatment as indicated above. An asthmatic may have improved control over optimized pharmacological control via additive effects of SYMP and/or PSYMP modulation of the bronchial response, acutely or chronic, independent of pharmacological management.

This disclosure is not intended to be limited to the scope of the particular forms set forth, but is intended to cover alternatives, modifications, and equivalents of the variations described herein. Further, the scope of the disclosure fully encompasses other variations that may become obvious to those skilled in the art in view of this disclosure. The scope of the present invention is limited only by the appended claims.

The particular embodiments disclosed above are illustrative only, as the invention may be modified and practiced in different but equivalent manners apparent to those skilled in the art having the benefit of the teachings herein. Embodiments of the present invention disclosed and claimed herein may be made and executed without undue experimentation with the benefit of the present disclosure. While the invention has been described in terms of particular embodiments, it will be apparent to those of skill in the art that variations may be applied to systems and apparatus described herein without departing from the concept, spirit, and scope of the invention. Examples are all intended to be non-limiting. It is therefore evident that the particular embodiments disclosed above may be altered or modified and all such variations are considered within the scope and spirit of the invention, which are limited only by the scope of the claims.

1-117. (canceled)

118. A device for transdermal stimulation of a peripheral nerve, including the auricular branch of the vagus nerve, the device comprising:
(a) a control unit configured to generate an electrical signal at a frequency in a frequency range from 1 kHz to 50 kHz; and
(b) at least one housing designed to be fitted on or in a patient's ear, the housing carrying at least one pair of electrodes, the at least one pair of electrodes being coupleable to the control unit to deliver the electrical signal to a neural structure of the patient's ear, including an auricular branch of the patient's vagal nerve, via the at least one pair of electrodes.

119. The device of claim 118 wherein the control unit is positioned within the housing.

120. The device of claim 118 wherein the at least one housing is a first housing designed to fit one of the patient's ears, and wherein the device further includes a second housing designed to fit the other of the patient's ears.

121. The device of claim 118 wherein the electrical signal has electrical pulses with pulse widths in a pulse width range from 1 microsecond to 500 microseconds.

122. The device of claim 118 wherein the electrical signal has an amplitude in an amplitude range from 0.1 mA to 20 mA.

123. The device of claim 118 wherein the frequency range is from 10 kHz to 25 kHz.

124. The device of claim 118 wherein the frequency is 20 kHz.

125. The device of claim 118 wherein the control unit is configured to deliver the electrical signal for up to one hour, and no more than twice daily.

126. The device of claim 118 wherein the control unit is configured to halt the electrical signal for a period of from one day to one month.

127. The device of claim 118 wherein the electrical signal is a patient-imperceptible electrical signal.

128. The device of claim 118 wherein the electrical signal is a first electrical signal, and wherein the frequency is a first frequency, and wherein the control unit is configured to:
(a) deliver a first portion of the first signal at a first frequency and a second portion of the first signal at a second frequency different than the first frequency, or
(b) both the first signal at the first frequency, and a second signal at the second frequency, or
(c) both (a) and (b).

129. A method of treating a patient, comprising:
generating a pulsed electrical signal having a frequency in a frequency range from 1 kHz to 50 kHz, a pulse width in a pulse width range from 1 microsecond to 500 microseconds, and an amplitude in an amplitude range from 0.1 mA to 20 mA; and
transcutaneously directing the electrical signal to a neural structure of the patient's ear, including an auricular branch of a patient's vagal nerve, via the skin of a target portion of the patient's ear.

130. The method of claim 129 wherein the frequency range is from 10 kHz to 25 kHz.

131. The method of claim 129 wherein the frequency is 20 kHz.

132. The method of claim 129 wherein the pulse width range is from 10 microseconds to 50 microseconds.

133. The method of claim 129 wherein the amplitude range is from 1 mA to 5 mA.

134. The method of claim 129 wherein delivering the electrical signal includes delivering the electrical signal for up to one hour, no more than twice daily.

135. The device of claim 129, further comprising halting the electrical signal for a period of from one day to one month.

136. The method of claim 129 wherein the electrical signal is delivered without inducing a perceptible sensation in the patient.

137. The method of claim 129, further comprising adjusting at least one parameter of the electrical signal in response to sensed feedback from the patient.

138. The method of claim 129 wherein treating the patient includes treating the patient for arthritis.

139. The method of claim 129 wherein directing the electrical signal includes directing the electrical signal via at least one electrode positioned at the cymba concha of the patient's ear.

140. A method of treating rheumatoid arthritis in a human patient, comprising:
(a) positioning at least two electrodes at the patient's skin, on or in the patient's ear; and
(b) treating the rheumatoid arthritis by transcutaneously delivering an electrical signal to an auricular branch of the patient's vagal nerve via the at least two electrodes, the electrical signal having a frequency in a frequency range from 1 kHz to 50 kHz.

141. The method of claim 140 wherein treating the rheumatoid arthritis is performed without use of a pharmaceutical in conjunction with the delivering the electrical signal.

142. The method of claim 140 wherein the electrical signal is delivered without inducing a perceptible sensation in the patient.

143. A method of treating a human patient, comprising:
(a) positioning at least two electrodes at the patient's skin, on or in the patient's ear;
(b) transcutaneously delivering an electrical signal to a neural structure of the patient's ear, including an auricular branch of the patient's vagal nerve, via the at least two electrodes, the electrical signal having a frequency in a frequency range from 1 kHz to 50 kHz, and being imperceptible to the patient; and
(c) administering an effective amount of a pharmaceutical to the patient, in conjunction with directing the electrical signal to the auricular branch of the patient's vagal nerve

144. The method of claim 143 wherein the pharmaceutical is selected from the group consisting of abatacept, adalimumab, adalimumab-atto, anakinra, certolizumab, etaneracept, etanercept-szzs, golimumab, infliximab, infliximab-dyyb, rituximab, tocilzumab, tofacitinib, methotrexate and an NSAID.

145. The method of claim 143 wherein administering the pharmaceutical includes administering a reduced dosage of the pharmaceutical to the patient compared to a treatment regimen for the patient that does not include transcutaneously delivering the electrical signal.

146. The method of claim 143 wherein:
the electrical signal is delivered to the patient to address the patient's arthritis,
the frequency of the electrical signal is from 10 kHz to 25 kHz,
the electrical signal has pulses with pulse widths in a pulse width range from 10 microseconds to 50 microseconds, and
an amplitude of the electrical signal is in an amplitude range from 0.1 mA to 15 mA.

147. The method of claim 143 wherein the electrical signal is halted after a session period of 15 minutes, and wherein the frequency of the electrical signal is 20 kHz.
